(19) United States
(12) Patent Application Publication
(10) Pub. No.: US 2025/0144603 A1
(43) Pub. Date: May 8, 2025

(54) CATALYST COMPOSITION OF MATTER FOR PRODUCTION OF PERCARBOXYLIC ACIDS

(71) Applicant: SYMBIENT ENVIRONMENTAL TECHNOLOGIES, MISSISSAUGA (CA)

(72) Inventors: WILLIAM KEVIN O'KEEFE, GEORGETOWN (CA); SABA KARIMI, MISSISSAUGA (CA)

(21) Appl. No.: 18/837,313

(22) PCT Filed: Feb. 9, 2023
(86) PCT No.: PCT/CA2023/050170

§ 371 Date: Aug. 9, 2024

Related U.S. Application Data
(60) Provisional application No. 63/308,183, filed on Feb. 9, 2022.

(51) Int. Cl. B01J 23/648; B01J 21/04; B01J 23/20; B01J 23/30; B01J 23/44; B01J 23/50; B01J 31/08; B01J 31/36; B01J 35/40; B01J 35/61; C07C 409/24

(52) U.S. Cl. CPC B01J 23/6484; B01J 21/04; B01J 23/20; B01J 23/30; B01J 23/44; B01J 23/50; B01J 31/08; B01J 31/36; B01J 35/40; B01J 35/612; B01J 35/613; B01J 35/615; B01J 35/617; B01J 35/618; C07C 409/24; B01J 2231/70; B01J 2531/002

(57) ABSTRACT

The present disclosure provides a catalyst for the production of percarboxylic acids which includes a co-catalyst 1 being either a transition metal oxide or carbonaceous compound and a second co-catalyst 2, distinct from co-catalyst 1 and being comprised of at least one transition metal, transition metal oxide, transition metal carbide or transition metal nitride. The combination of these co-catalysts provides the necessary kinds of catalytic active sites for both the chemisorption and activation of formic acid as well as active sites for the generation of surface active oxygen species. The combination of these co-catalyst materials results in the synergistic benefits of enhancement in catalytic activity for the production of the peracid as well as improved catalyst stability.

### FIELD

The present disclosure pertains to a catalyst for the production of percarboxylic acids, in particular performic acid (PFA) for use as a disinfectant in water treatment, medical device disinfection, industrial effluent treatment and food processing equipment disinfection or for the in situ production of PFA for use as a chemical feedstock, intermediate or oxidizing agent in the synthesis and production of specialty chemicals, such as the production of value added products from the epoxidation of vegetable oil.

### BACKGROUND

To date, 90% of wastewater treatment plants in North America continue to use chlorine based chemical disinfectants as the primary means of disinfection (Ref. 1). However, due to its toxicity, the treated effluent often must be neutralized to a significant extent before discharge to the environment, adding cost and complexity to the process. The use of chlorine as a biocide to disinfect water dates back to 1850. Despite its maturity as a technology, it is now well known, and the subject of several scientific studies including those by the World Health Organization and other regulatory bodies, that chlorine based disinfection results in the formation of undesirable and potentially hazardous disinfection byproducts (DBPs), such as haloquinones, haloamines, halonitriles, n-chloramines, haloacetamides, nitrosamines and so on created from the reaction of chlorine-containing compounds with natural organic matter already present in water. Hundreds of DPBs have been detected in drinking water, some of which are known to be carcinogenic. (Ref. 2) Consequently, there is significant motivation to move away from the incumbent chlorine-based disinfection technology to a more sustainable alternative, in all stages of water treatment, not just drinking water treatment, as a result of the circular and interconnected nature of water as a resource. Emerging environmental and regulatory drivers, such as WSER regulation (Canada) mandating a reduction in chlorine usage in water treatment, further add pressure to adopt an alternative technology. However, cost, both capital and operating, remain the main reason alternative technologies have not yet been adopted.

PFA is an emerging disinfectant for use in water treatment, food processing and medical device disinfection and has been the subject of numerous disinfection efficacy studies including pilot scale studies. PFA provides rapid and efficient disinfection of bacteria and viruses even at low doses and at low temperatures, which is important for water treatment applications (Ref. 3). A techno-economic lifecycle analysis of PFA, hypochlorite and peracetic acid disinfectants for water treatment application concluded that PFA was the most potent for the inactivation of the pathogens and had the lowest environmental impact of the three disinfectants (Ref. 4). PFA does not produce disinfection byproducts and will spontaneously decompose within 12 hours into non-toxic byproducts, specifically water and carbon dioxide.

Due to its instability, PFA must be produced at the point of use. PFA is produced from the reaction of hydrogen peroxide with formic acid in a batch or semi-batch reactor using a homogeneous catalyst, typically a strong mineral acid like sulphuric acid, nitric acid, phosphoric acid or other strong acids (Ref. 5). Since the reaction is equilibrium limited, the product after some time is an equilibrium mixture of PFA with unreacted formic acid and hydrogen peroxide and the homogeneous catalyst. The state-of the art process for producing PFA for water treatment is the Kemira (Helsinki, Finland) KemConnect™ DEX process. This process combines formic acid and hydrogen peroxide in the presence of a homogeneous catalyst in a semi-batch reactor, resulting in an equilibrium mixture of 13.5 wt. % PFA, 30.9 wt. % unreacted formic acid and 20 wt. % unreacted hydrogen peroxide (Ref. 6).

The production of peracids compounds such as peracetic acid, whose syntheses are equilibrium limited, can be produced by reactive distillation or catalytic distillation, whereby yields far greater than the equilibrium conversion are possible due to the continuous removal of a product by distillation from the reaction zone, thereby shifting the reaction in favor of product formation in accordance with Le Chatelier's Principle. It has been shown experimentally that conversion as high as essentially 100% can be achieved for reactions that would otherwise be equilibrium limited (Refs. 7, 8). Pohjanvesi et al. (Ref. 9) have proposed a reactive distillation process for the production of percarboxylic acids, particularly peracetic acid, whereby a homogeneous catalyst is fed continuously to the reaction medium and the product continuously distilled, with a peracetic rich distillate stream, and the inventors claim this process can be used for the production of any percarboxylic acid.

The presence of the strong mineral acid in the product can cause corrosion issues in the downstream process piping and equipment (Refs. 10, 11). Since formic acid is acidic, the synthesis of PFA is autocatalytic and can proceed in the absence of a catalyst, albeit at a much lower rate, such that the autocatalytic reaction is not a commercially viable route to produce PFA. To avoid the use of a mineral acid catalyst, Balasubramanian et al. (Ref. 10) proposed conducting the production of PFA in the absence of a catalyst in a flow reactor but using heat to accelerate the reaction. However, heating the water in a flow system is an energy intensive process. More importantly, heating PFA, formic acid and hydrogen peroxide without failsafe controls, is generally not advised as it is known that the reactants formic acid, hydrogen peroxide and the product PFA are energetic materials which can potentially explode when heated. Heating hydrogen peroxide and the product performic acid will result in their decomposition, significantly lowering the process yield. Moreover, the thermal decomposition of PFA and hydrogen peroxide are both strongly exothermic reactions which can further facilitate their decomposition, potentially resulting in runaway reactions (Ref. 12). Ebrahimi et al. (Refs. 13, 14) found that in the presence of a sulphuric acid catalyst that the decomposition of PFA becomes the dominant reaction above 40° C. In short, the synthesis of PFA without a catalyst is not practical commercially due to slow reaction rates.

Aksela and Matilla (Ref. 11) proposed using a homogeneous catalyst other than a strong mineral acid catalyst, whereby the catalyst was comprised of a carboxylic acid ester. However, when using a homogeneous catalyst, the contact time of PFA with the catalyst cannot be controlled, causing undesirable consecutive reactions further lowering the process yield. Moreover, a significant disadvantage of using a homogeneous catalyst is that the catalyst itself is a consumable in this process as it is introduced in a continuous feed stream and becomes part of the product being discharged from the process, significantly contributing to the operating cost of the process.

The use of a heterogeneous (solid) catalyst can obviate the aforementioned undesirable characteristics of the homogeneous catalyst. A solid-state catalyst would allow facile separation and recovery of the catalyst from the reactant mixture or in the case of when immobilized in a flow reactor, would allow more precise control of contact times of the reactants and products with the catalyst. Chemical reactions would occur at the liquid-solid interface at the catalyst surface, where the chemistry can be precisely controlled by molecularly engineered catalyst active sites to ensure high selectivity towards the desired reaction product, PFA. However, a robust solid-state catalyst for the synthesis of PFA does not exist in the prior art.

Strongly acidic solid state ion exchange resin catalysts have been shown to be active in facilitating the synthesis of PFA from formic acid and hydrogen peroxide (Ref. 14). However, ion exchange resins are not moisture tolerant; and are known to deactivate in the presence of water (Refs. 15, 16, 17). A lack of moisture tolerance and leaching of acidity is a known issue with solid state acid catalysts. Aside from lack of moisture tolerance and leaching of acidity, a heterogeneous catalyst must be stable in the relatively harsh reaction environment which includes concentrated hydrogen peroxide. The current inventors found that commercially available Amberlyst 15 strongly acidic cation exchange resin rapidly deactivated, losing approximately 70% of its activity within the first 24 hours of operation in a semi pilot scale catalytic distillation reactor (see Example 1). In a separate experiment (see Example 2), the inventors found that Amberlyst 15 had lost more than half of its activity when exposed to a relatively low concentration of hydrogen peroxide, 10 wt % (aq) in a beaker over approximately 72 hour period.

Tanabe (Ref. 18) summarizes numerous solid state acid catalysts and their application and points to hydrated niobium oxide as an unusually acidic and moisture tolerant heterogeneous catalyst. However, the current inventors found that niobium oxide catalysts, although showing good initial activity for PFA synthesis, deactivated relatively rapidly upon exposure to hydrogen peroxide, precluding their use for potential commercial application (see Example 2).

A robust heterogeneous catalyst would be a significant advancement in the art as it would enable the use of flow reactors, slurry reactors, fluidized bed reactors, catalytic distillation reactors and catalytic membrane reactors for the production of PFA, obviating the need to use a liquid catalyst, resulting in improved process yield and efficiency and improved process economics. It is an object of the current invention to mitigate one or more of the aforementioned deficiencies of the homogeneous and heterogeneous catalysts described in the prior art. The inventors disclose herein, a heterogeneous catalyst which is very active for the synthesis of PFA from formic acid and hydrogen peroxide and is also demonstrated to be stable within this reaction environment.

### SUMMARY

The present disclosure provides a catalyst for the production of a percarboxylic acid. The catalyst comprises a mesoporous catalyst material having an external surface and internal pores and having inherent surface acidity or surface basicity, said mesoporous catalyst material being in a form of i) a particle or extrudate ranging in size from about 0.2 to about 25 mm, or ii) a monolith, or iii) one or more thin films having a thickness in a range from about 1 to 1000 μm. The mesoporous catalyst material has a specific surface area ranging from about 1 to about 10,000 m²/g. The catalyst includes a co-catalyst comprised of any one or combination of WO₃ and Ag₂O dispersed as particles or thin films onto the internal surface area and/or external surface area of the mesoporous catalyst material.

The mesoporous catalyst material may have a specific surface area in a range from about 100 to about 600 m²/g, or a specific surface area in a range from about 150 to about 600 m²/g.

The mesoporous catalyst material may possess either inherent surface acidity or inherent surface basicity is characterized in that it
i) provides active sites which can participate directly in the perhydrolysis reaction due to the inherent surface acidity or basicity of the mesoporous catalyst material or other unique imperfections, surface defects, functional groups or features, or
ii) creates adlineation sites at phase boundaries between the mesoporous catalyst material and the dispersed co-catalyst, or
iii) interacts with the co-catalyst to generate weak or strong metal support interactions at the reaction conditions, or
iv) any combination of i), ii) or iii).

The mesoporous catalyst material may possess an inherent surface acidity characterized by a Hammet acidity of less than 3.

The mesoporous catalyst material may be any one of Al₂O₃, WO₃, MgO, Nb₂O₅, TiO₂, MoO₃, Fe₂O₃, FeO, V₂O₅, MgO, ZnO, Y₂O₃, Sc₂O₃, ZrO₂, WO₃, W₂O₅, CeO₂, Ce₂O₃, Ta₂O₃, ZrWₓO_{y} (wherein x is 2 and y is 0.5 to 8), SnO₂, activate carbon, graphite and mixtures thereof.

The mesoporous material may be Al₂O₃.

The mesoporous material may be an aluminosilicate with the general formula M^{n*}_{x/n}[(Al₂O₃)*(SiO₂)ₓ]*yH₂O, wherein M is a metal cation and n is the charge on the metal cation, typically ranging from 1 to 3, and x represents the silica to alumina ratio varying over a broad range from 1 to 500.

The mesoporous catalyst material may be an acidic ion exchange resin or a basic ion exchange resin

The mesoporous catalyst material may be an acidic or basic polymer bead.

The mesoporous catalyst material may be a hydrotalcite with the general formula [M²⁺₁₋ₓM³⁺ₓ(OH)₂]^{x+}Aⁿ⁻_{x/n}*mH₂O wherein M²⁺ and M³⁺ are divalent and trivalent transition metal cations respectively and Aⁿ⁻ is an exchangeable anion, wherein x ranges from 0.2 to 0.4, and n is 1 or 2. The mesoporous catalyst material may be a mixture of hydrotalcite materials.

The mesoporous catalyst material may be a semiconductor selected from the group consisting of Ta₂O₅, NaTaO₃, SrTiO₃, CdS, InVO₄, Mn₂O₃, Bi₂WO₆, BiVO₄, Ga₂O₃ and ZnGa₂O₄.

The any one or combination of co-catalyst WO₃ and Ag₂O may be dispersed as sub-micron sized particles.

The any one or combination of co-catalyst WO₃ and Ag₂O are dispersed as films having a thickness in a range from about 10 to about 300 μm.

The mesoporous catalyst material may be in the form a particle or extrudate having a size in a range from about 0.2 to about 25 mm.

The mesoporous catalyst material may be in the form a particle or extrudate having a size in a range from about 3 mm to about 11 mm.

The mesoporous catalyst material may be in the form a film having a thickness in a range from about 1 to about 1000 μm.

The mesoporous catalyst material may be in the form a film having a thickness in a range from about 5 to about 300 μm.

The mesoporous catalyst material may be in the form a film having a thickness in a range from about 10 to about 200 μm.

The percarboxylic acid may be performic acid.

The present disclosure also provides a catalyst for the production of percarboxylic acids, comprising:
a mesoporous catalyst carrier material having an external surface and interior pores extending therethrough;
at least first and second distinct co-catalyst materials which are dispersed onto the external surface and/or into the interior pores of the mesoporous catalyst material either as particles or in one or more thin films;
the first co-catalyst being derivatized to produce metal peroxo groups;
the at least second co-catalyst selected to interact with said first co-catalyst by providing active sites for the chemisorption of the parent carboxylic acid and or by providing surface features to facilitate the surface mobility and migration of surface active oxygen species.

The metal peroxo groups are capable of storing and releasing surface active oxygen to the catalyst surface. The product, peracid is created from the concerted addition of surface active oxygen species to the parent acid, which is chemisorbed on an active site of co-catalyst 2, which then desorbs from the active site of co-catalyst 2, thereby releasing the product from the catalyst while regenerating the active sites for the next catalytic cycle. Similarly, the presence of concentrated hydrogen peroxide regenerates the metal peroxo group, for the next catalytic cycle.

The first co-catalyst derivatized to produce metal-peroxo groups may be any one or combination of Nb₂O₅, SiO₂, TiO₂, MoO₂, Al₂O₃, Fe₂O₃, V₂O₅, MgO, ZnO, Y₂O₃, Sc₂O₃, ZrO₂, W₂O₅, WO₃, CeO₂ and Ta₂O₃.

The co-catalyst derivatized to produce metal peroxo groups may be Nb₂O₅.

The at least a second co-catalyst may be any one or combination of transition metals or oxides of transition metals from the group consisting of Ag, Pt, Pd, W, Ni, Rh, Ru, Au, Ir, Os, Re, Ta, Hf, Zr, Ti, V, Cr, Mo, Mn, Co, Zn, Fe and Cu.

The at least a second co-catalyst may be any one or combination of metal carbides of the transition metals selected from the group {M, V, W, Ti, Co} or metal nitride of the transition metals selected from the group consisting of W, Mo, Ti, V and Nb.

The at least a second co-catalyst may be PdO.

The mesoporous catalyst carrier may be
selected from the group consisting of SiO₂, Al₂O₃, TiO₂, MgO, WO₃, MnO₂, MoO₂, ZnO, Fe₂O₃, V₂O₅, Y₂O₃, Sc₂O₃, ZrO₂, CeO₂, Ce₂O₃ and Ta₂O₃, or
is an aluminosilicate with the general formula Mⁿ⁺_{x/n}[(Al₂O₃)*(SiO₂)ₓ]*yH₂O, wherein M is a metal cation and n is the charge on the metal cation ranging from 1 to 3, and x represents the silica to alumina ratio which can vary over a broad range from 1 to 500; or
is a hydrotalcite material with the general formula [M²⁺₁₋ₓM³⁺ₓ(OH)₂]^{x+}Aⁿ⁻_{x/n}*mH₂O wherein M²⁺ and M³⁺ are divalent and trivalent transition metal cations respectively and Aⁿ⁻ is an exchangeable anion, wherein x ranges from 0.2 to 0.4, n is typically 1 or 2; or any combination thereof.

The mesoporous catalyst carrier may be either SiO₂ or Al₂O₃.

A further understanding of the functional and advantageous aspects of the present disclosure can be realized by reference to the following detailed description and drawings.

### BRIEF DESCRIPTION OF THE DRAWINGS

Embodiments will now be described, by way of example only, with reference to the drawings, in which:

FIG. 1 shows Scheme 1 illustrating the deactivation of a solid-state acid transition metal oxide catalyst due to loss of Bronsted acidity accompanied with the reaction of its acidic surface hydroxyl groups with hydrogen peroxide to form metal-peroxo groups.

FIG. 2 shows Scheme 2 illustrating the activation of formic acid for reaction by its chemisorption on a Lewis acid site adjacent to metal-peroxo active site which acts as a oxygen donor followed by the desorption of the product PFA resulting in the regeneration of the Lewis acid site and illustrating the regenerated metal peroxo group due to the presence of hydrogen peroxide (i.e., Scheme 1).

FIG. 3 shows a plot of the PFA productivity (mmol/hr*g_{cat}) using commercially available Amberlyst™ 15 catalyst at various weight hourly space velocities (WHSV) when in the fresh (unused) condition and after exposure to 10 wt % H₂O₂ (aq) for approximately 72 hours.

FIG. 4 shows the high resolution Pd 3d XPS spectrum for the PdO/Nb₂O₅/Al₂O₃ catalyst.

FIG. 5 shows the high resolution Nb 3d XPS spectrum for the PdO/Nb₂O₅/Al₂O₃ catalyst.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described so as to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps, or components.

As used herein, the term “exemplary” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

As used herein, the phrase “catalyst carrier” refers to a pellet, extrudate, irregularly shaped particle, monolith or film or coating that has a high specific surface area, ranging from about 1 to about 10,000 m²/g, but more commonly from 100 to 500 m²/g, and having a sufficiently developed internal pore structure, which allows the dispersion of sub-micron sized particles including nanoparticles of the catalytic phase or phases onto the surface of the carrier by using deposition methods such as impregnation, grafting, templating, atomic layer deposition, chemical vapour deposition, surface attaching reactions or other techniques that would be known to one skilled in the art.

A catalyst carrier may be an inorganic ceramic support comprised of one or more transition metal oxides such as Al₂O₃, SiO₂, TiO₂, CeO₂, V₂O₅ and so on or may be comprised of a carbonaceous material such as a polymer or activated carbon for example. In reference to the pore size distribution of the catalyst carrier, micropores are those pores which have a characteristic dimension of 2 nm or less while macropores are large pores which are greater than 50 nm in characteristic dimension. Mesopores are those which have a characteristic dimension ranging from 2 to 50 nm. It is known to one skilled in the art that solids with mesoporous structure are useful as catalyst carriers, since the pore size is not so small to cause undesirable issues related to the impedance of the diffusion of chemicals and or cause issues related to catalyst fouling but are also sufficiently small to generate a very high interfacial area to promote the chemical reaction.

In this disclosure we refer to the catalyst carrier as a mesoporous material, without loss of generality. The description is not meant be limiting to exclude carriers which exhibit either micropores or macropores and it is understood that certain catalyst carriers with micropores and macropores may have utility for this intended purpose. Particles and extrudates are often used as catalyst carriers, however it is known to one skilled in the art that significant internal mass transfer limitations arise when catalyst carrier particles smaller than about 3 mm are used. However, the particle size of the catalyst carrier can also affect flow conditions and pressure drop in a reactor. Commercial catalyst carriers typically range in size from having a characteristic dimension of 3 to 11 mm, however some microspheres and micro-extrudates having 0.3 to 3.5 mm in characteristic dimension are used in certain applications.

As used herein, the phrase “co-catalyst” refers to a functional catalytic material, typically in the form of a nanoparticle or larger crystallite or a single atom or functional group or as a thin film that is active or promotes the desired chemical reaction by providing active sites for the chemisorption of reactants and stabilization of reaction intermediates, and or promotes the catalytic turnover of a second catalyst phase and or promotes the stability and robustness of the catalyst system. A co-catalyst is either a chemically distinct phase or material that is distinguishable from both the catalyst carrier and other co-catalysts present or is a chemical dopant that can modify the structure and or properties of the other catalytic phases present. The co-catalyst is deposited in a manner to result in a high dispersion whereby the co-catalyst is deposited onto the catalyst carrier in the form of very small particles such as sub-micron sized crystallites and nanoparticles which have very high surface area to volume ratio and physicochemical properties that are distinct from their corresponding bulk materials.

The co-catalyst phase is typically deposited at sub-monolayer coverage, or up to monolayer coverage, such that these unique properties are preserved, as would be known to one skilled in the art.

The catalyst carrier is a mesoporous material in either the form of a particle or extrudate or powder or a thin film or coating, which may be inert or may serve as a co-catalyst (i.e., co-catalyst #1) by exhibiting significant surface acidity or basicity or other structural defects, features and imperfections that can facilitate catalytic turnover and as would be known to one skilled in the art and may be selected from a list of metal oxides such as {SiO₂, Al₂O₃, TiO₂, WO₃, W₂O₅, MoO₃, MgO, ZnO, Fe₂O₃, Y₂O₃, Sc₂O₃, ZrO₂, CeO₂, Ta₂O₃ etc.} or any combination thereof or may be a zeolite or combination of zeolites or may be a polymeric material or combination or blend of polymeric materials or may be a carbonaceous carrier material such as activated carbon, carbon black, graphene, graphene oxide, graphite, or combinations thereof. The catalyst carrier may be a large particle (e.g., 3 to greater than 11 mm in characteristic dimension), or a micro-sphere or equivalent particle with characteristic dimension ranging from 0.3 to 3 mm, which can be immobilized inside a chemical reactor, or the catalyst carrier may be in powder form, which can be used as an active ingredient in a catalytic coating or used as a slurry.

The present disclosure provides a heterogeneous catalyst comprised of two or more co-catalysts, which work synergistically to promote the perhydrolysis reaction to produce performic acid from hydrogen peroxide and formic acid and to promote the stability of the catalyst in the reaction environment. Either one or more co-catalyst exhibit sites which 1) generate surface active oxygen species from the dissociative chemisorption of hydrogen peroxide and 2) exhibit active sites for the chemisorption and activation of formic acid to react with surface active oxygen species to produce the product PFA. The two or more co-catalyst phases are distinct chemically from each other. Their synergistic interaction results in chemical reaction rates and selectivity for the production of PFA that are greater than would be observed from either co-catalyst operating in isolation from the other and whose long term stability in the reaction environment is improved compared to the long term stability of either co-catalyst operating in isolation of the other.

The catalyst exhibits excellent activity for the synthesis of PFA and Is stable in the reaction environment, thereby enabling PFA synthesis in various reactor systems including batch, semi-batch or continuous stirred tank reactor (CSTR) wherein the catalyst may be either in a slurry or immobilized within the CSTR. The catalyst may be immobilized in a fixed bed flow reactor (FBR); catalytic distillation reactor; or immobilized within catalytic membrane reactor by deposition of catalytic phases or films into the membrane. The catalyst may be incorporated into a thin film coating the internals of a microfluidic reactor or may be used as fine particulate slurry within a fluidized bed reactor and so on. The catalyst may be dispersed onto large catalyst carrier particles or included as fine particulates within catalytic wash coats or sol gel films for dispersion onto monoliths or other structured packings, onto surfaces of reactor internals or onto membranes.

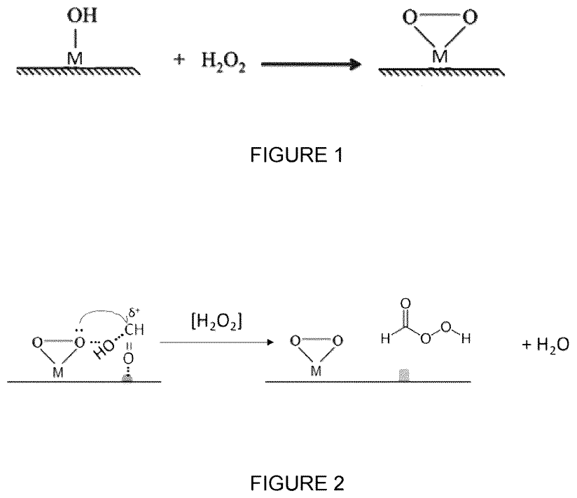

The current inventors have surmised that the deactivation of solid acid catalysts containing transition metals, such as hydrated Nb₂O₅, in the presence of hydrogen peroxide, is due to the formation of metal-peroxo groups (Scheme 1 shown in FIG. 1) resulting in a loss of Bronsted acidity, specifically the loss of acidic hydroxyl groups of the solid acid catalyst which are the active sites for the perhydrolysis reaction to produce PFA from hydrogen peroxide and formic acid. This phenomenon of the formation of metal-peroxo groups has been observed previously on transition metal oxides and is known. In addition, the reaction of metals with hydrogen peroxide in some cases can lead to the formation of soluble metal-peroxo complexes resulting in leaching of the transition metal from the catalyst.

It has been reported that metal-peroxo groups can serve as oxygen storage and oxygen donor sites for catalytic oxidation reactions, which was demonstrated by Cardoso et al. (Ref. 19) for the oxidation of an organic dye (methylene blue) from the tungsten-peroxo group in the presence of H₂O₂. The active site (metal peroxo-group) is regenerated in situ due to reaction of the surface hydroxyl remaining after oxygen donation with hydrogen peroxide present in the system, thereby completing the catalytic cycle. However, in the case of PFA synthesis, the current inventors observed that the formation of metal-peroxo groups resulted in the irreversible deactivation of the solid acid catalyst, which was accompanied by a characteristic colour change of the catalyst from white to bright yellow (see Example 3).

Without wishing to be bound by theory, the inventors hypothesized that certain metal-peroxo groups would be thermodynamically stable in the presence of concentrated hydrogen peroxide and could serve as oxygen storage sites and facilitate the concerted insertion of oxygen to formic acid to yield PFA, provided that formic acid is first activated for reaction by its chemisorption onto an adjacent active site associated with a co-catalyst, for example one exhibiting Lewis acidity (see Scheme 2 shown in FIG. 2). After the reaction and desorption of the product performic acid, the metal-peroxo group is regenerated due to the presence of concentrated hydrogen peroxide as described by Cardoso et al. (Ref. 19) making this nanostructure a regenerable catalyst active site (i.e., co-catalyst #1). Therefore, the invention provides an alternative reaction mechanism to facilitate the perhydrolysis synthesis reaction of PFA from formic acid and hydrogen peroxide, namely via the concerted addition of surface active oxygen to chemisorbed formic acid.

Through the course of experimentation, the current inventors screened numerous catalyst systems comprised of co-catalysts dispersed onto a mesoporous catalyst carrier whereby the first catalyst (co-catalyst #1) was a nanostructured solid acid catalyst which had been derivatized by exposure to hydrogen peroxide to convert its acidic surface hydroxyl groups to metal-peroxo groups and the second catalyst (co-catalyst #2) being either a transition metal or metal oxide nanoparticle or larger crystallite. The results after several months of study were negative, in that the catalyst systems investigated either showed low activity or showed promising initial activity but deactivated significantly in stability tests. Eventually, the current inventors unexpectedly found a catalyst comprised of palladium oxide combined with nanostructured niobium oxide dispersed at sub monolayer coverage onto a mesoporous Al₂O₃ catalyst carrier which gave a positive result.

The resulting catalyst was found to be more active than Amberlyst™ 15 for PFA synthesis at a comparable space velocity in a fixed bed reactor. Moreover, the catalyst was found to be stable in stability tests and maintained its activity after deliberate exposure to concentrated hydrogen peroxide for 48 hours (see Example 4). In addition, the catalyst was studied in a semi-pilot plant test and only showed a slight decrease in activity by about 11% upon completion of the study; (it is known to one skilled in the art that some catalyst deactivation should be expected relative to the fresh condition, after a fresh catalyst is first put into application). The inventors found experimentally that a PdO/Al₂O₃ catalyst with the same mass fraction of Pd as the aforementioned PdO/Nb₂O₅/Al₂O₃ catalyst was also active for the synthesis of PFA but showed lower activity and poor stability in contrast to the PdO/Nb₂O₅/Al₂O₃ catalyst (see Example 6).

### First Embodiment #1

In the Embodiment #1, two or more co-catalysts, which are primarily responsible for the catalytic properties of the catalyst are dispersed onto a catalyst carrier whose surface may be either inert or exhibits surface acidity or basicity such as a carbonaceous material including a polymer or activated carbon, or is comprised of an inorganic material such as an aluminosilicate (zeolite) or is comprised of one or more transition metal oxides. More preferably the catalyst carrier is comprised of one or more mesoporous transition metal oxides selected from the group {Nb₂O₅, SiO₂, TiO₂, MoO₂, Al₂O₃, Fe₂O₃, V₂O₅, MgO, ZnO, Y₂O₃, Sc₂O₃ ZrO₂, W₂O₅, WO₃, CeO₂, Ta₂O₃}. Most preferably, the catalyst carrier is a mesoporous Al₂O₃ with a specific surface area of at least 150 m²/g.

Co-catalyst #1 may be comprised of an inorganic material such as one or more zeolites, one or more transition metal oxides, with its surface either inert, acidic or basic. Alternatively, co-catalyst #1 may be comprised of a carbonaceous material such as a carbon nanotube, graphene or graphene oxide. Co-catalyst #1 may be a dopant, such as a halogen, a lanthanide, an alkali metal or alkali earth whose purpose is to modify the catalytic properties of co-catalyst #2. More preferably co-catalyst #1 is a cluster comprised of one or more transition metal oxides selected from the group {Nb₂O₅, SiO₂, TiO₂, MoO₂, Al₂O₃, Fe₂O₃, V₂O₅, MgO, ZnO, Y₂O₃, Sc₂O₃, ZrO₂, W₂O₅, WO₃, CeO₂, Ta₂O₃}which can be derivatized to produce stable metal-peroxo groups. Most preferably co-catalyst #1 is comprised of nanostructured Nb₂O₅ nanoparticles exhibiting significant Bronsted acidity, which can be derivatized by reaction with hydrogen peroxide to produce Nb-peroxo groups, after dispersion onto the catalyst carrier at sub-monolayer coverage.

Co-catalyst #2 is either a sub-micron sized transition metal cluster, a sub-micron sized transition metal oxide cluster or a cluster comprised of an alkaline or alkali earth, such Ca or Sc compounds. Alternatively, co-catalyst #2 may be a metal carbide such as {MoC, Mo₂C}), VC, W₂C, WC, TiC, CoC₂,} or metal nitride such as {W₂N, Mo₂N, TiN, VN, NbN} or bimetallic carbide or nitride such as {Ni₂Mo₃N}. More preferably, co-catalyst #2 is transition metal nanoparticle, nanowire or transition metal oxide nanoparticle where the transition metal is selected from {Ag, Pd, Pt, W, Cu, Ni, Co, Rh}. Most preferably, co-catalyst #2 is comprised of sub-micron sized palladium oxide particles.

Co-catalyst #1 may be deposited first onto the catalyst carrier; alternatively, co-catalyst #2 may be deposited first onto the catalyst carrier or both co-catalyst #1 and #2 may be deposited simultaneously onto the catalyst carrier following synthesis techniques that would be known to one skilled in the art such as co-precipitation methods. Without being construed as limiting, the invention is illustrated in Example 4, through the example of co-catalyst #1 being Nb₂O₅ which is first deposited onto the catalyst carrier, Al₂O₃, then derivatized by reaction with hydrogen peroxide to produce Nb-peroxo groups, followed by the deposition of co-catalyst #2, PdO onto the system of Nb₂O₅/Al₂O₃ resulting in a PdO/Nb₂O₅/Al₂O₃ catalyst. Although Al₂O₃ exhibits Lewis acidity and has some activity for PFA synthesis, in the form of this catalyst, the catalytic properties are governed by co-catalyst #1 and co-catalyst #2 phases.

The mass fraction of Nb₂O₅ after deposition onto the SiO₂ catalyst carrier should give 0.1 to 1.5 monolayer coverage. More preferably, mass fraction of Nb₂O₅ after deposition onto the catalyst carrier should give 0.2 to 1.2 monolayer coverage. Most preferably, Nb₂O₅ is deposited onto the carrier resulting in approximately 0.4 to 0.6 monolayer coverage.

The mass fraction of co-catalyst #1 required to achieve monolayer coverage, as known to one skilled in the art, depends on the specific surface area of the catalyst carrier and the geometrical dimensions of the nanomaterial deposited onto the carrier. In the preferred embodiment, the catalyst carrier is mesoporous Al₂O₃ and co-catalyst #1 is nanostructured Nb₂O₅. For catalyst carriers with specific surface areas of ranging from 150 to 300 m²/g, the mass fraction of Nb₂O₅, after deposition onto the catalyst carrier should range from 0.1 wt. % to 99.9 wt. %. More preferably the mass fraction of co-catalyst #1, after deposition onto the catalyst carrier should range from 5 wt. % to 35 wt. %. Most preferably the mass fraction of Nb₂O₅, after deposition onto the catalyst carrier should range from 9 wt. % to 18 wt. %.

After dispersion of co-catalyst #1 onto the catalyst carrier, co-catalyst #1 is derivatized to have surface hydroxyl groups converted to metal-peroxo groups by reaction with hydrogen peroxide solution. For example, the catalyst is immersed in 10 wt. % hydrogen peroxide for 2 hours at room temperature. Since the reaction is exothermic, lower concentrations and longer exposure times can be utilized. A water bath with substantial heat sink can be used to maintain temperature control in the sample, such as would be available with a rotary evapourator system.

The mass fraction of the transition metal of co-catalyst #2, in the final catalyst ranges from 0.01 to 99 wt. %. More preferably the co-catalyst #2 is PdO and the mass fraction of Pd in the resultant catalyst ranges from 0.01 to 5 wt. %. Most preferably, the mass fraction of Pd in the resultant catalyst ranges from 0.05 to 0.5 wt. %.

### Second Embodiment #2

Alternatively, in a second Embodiment #2, the catalyst carrier itself may have one or both of the requisite active sites either for the production of surface active oxygen species or for the chemisorption and activation of formic acid or may otherwise interact with the second co-catalyst in a manner which enhances the overall catalytic turnover or improves the stability of the catalyst system. Consequently, the catalyst carrier represents co-catalyst #1 and one or more additional co-catalysts are dispersed onto the catalyst carrier.

The present inventors found that some transition metal oxides without the Nb₂O₅ co-catalyst 1, were found to be both active and stable for PFA synthesis when mesoporous Al₂O₃ was used as a catalyst carrier. In particular, WO₃/Al₂O₃ and Ag₂O/Al₂O₃ catalysts were found to be both active and stable for PFA synthesis (Examples 7 and 8). Without wishing to be bound by theory, the inventors hypothesize that either these transition metal oxides (Ag₂O and WO₃) exhibit both of the requisite active sites by exhibiting active sites required to chemisorb and activate formic acid for reaction as well as exhibiting the active sites required to generate surface active oxygen species from the chemisorption of H₂O₂ or the Al₂O₃ support provides Lewis acid sites responsible for either the chemisorption and activation of formic acid or the dissociative chemisorption of hydrogen peroxide or both. The inventors found that the Al₂O₃ catalyst support alone was active for the synthesis of PFA, due to its intrinsic surface acidity, however, the combination of PdO and Nb₂O₅ phases or the use of WO₃ or Ag₂O phases dispersed onto the Al₂O₃ carrier significantly promote the PFA productivity.

In the Embodiment #2, the catalyst carrier is not chemically inert but rather does contribute substantively to the overall chemical reaction by exhibiting one or more kinds of active sites for either the dissociative adsorption of H₂O₂ to produce surface active oxygen species or the chemisorption of formic acid to activate it for reaction with surface active oxygen species to produce performic acid or the catalyst carrier otherwise has properties which promotes the performance of the other co-catalyst phases present through either a geometrical or electronic effect such as weak metal support interaction (WMSI) or strong metal support interaction (SMSI) or the catalyst carrier is responsible for the creation of unique adlineation sites, which are unique catalyst active sites at phase boundaries, or the catalyst carrier is responsible for the reaction of surface mobile spillover intermediates created by the co-catalyst phase whose reaction is completed at the active sites located on the catalyst carrier.

The catalyst carrier may be a mesoporous material which exhibits significant Lewis or Bronsted acidity or basicity such as a polymer or activated carbon. The catalyst carrier may be and inorganic material such as an aluminosilicate (zeolite) or a transition metal oxide. More preferably the catalyst carrier is comprised of one or more transition metal oxides selected from the group {Al₂O₃, TiO₂, WO₃, MoO₃, MgO, ZnO, Fe₂O₃, Y₂O₃, Sc₂O₃, ZrO₂, CeO₂, Ta₂O₃ etc.}Most preferably the catalyst carrier is a mesoporous material comprised of Al₂O₃ with a specific surface area of at least 150 m²/g.

### Example 1

Commercially available Amberlyst™ 15 (Sigma Aldrich 216380) was used as a catalyst for PFA production in a semi-pilot scale experiment in a catalytic distillation reactor.

Amberlyst™ 15 is the trade name of a strongly acidic, macroreticular cation exchange resin catalyst comprised of a polymeric bead composed of cross-linked divinylbenzene copolymers and functionalized with sulfonic acid groups giving rise to strong Bronsted acidity. The catalytic activity of Amberlyst™ 15 for PFA production was characterized previously in a fixed bed flow reactor for various reaction temperatures, feed concentrations of formic acid and hydrogen peroxide at room temperature and for a broad range of space velocities (WHSV) and for different concentrations of reactants. From this benchmarking data, an empirical model was created to predict the expected PFA production (mmol/hr*g_{cat}) as a function of space velocity when pristine Amberlyst™ 15 is used as a catalyst.

The catalytic distillation experiment was completed after 22.7 hours on stream. 5 random samples of the spent Amberlyst™ 15 catalyst were collected and their catalytic activity tested in a fixed bed reactor (FBR) at room temperature for inlet feed concentrations of 1 wt. % hydrogen peroxide and 1 wt. % formic acid. The results are summarized in (Table 1) The observed PFA production rates, when compared to the expected productivity for the fresh catalyst, shows that the Amberlyst™ 15 catalyst deactivated significantly, typically by greater than 70% loss of activity, with the exception of one sample (experiment 171) which did not appreciably deactivate.

| | | PFA [mmol/hr*g] | | Activity | |
|---|---|---|---|---|---|
| Expt. ID | WHSV [hr⁻¹] | Expected | Observed | (r [symbol] /r [symbol] ) | loss [%] |
| 169 | 136 | 12.84 | 3.45 | 0.269 | 73% |
| 170 | 182 | 15.95 | 4.82 | 0.302 | 70% |
| 171 | 136 | 12.84 | 14.51 | 1.130 | −13% |
| 172 | 343 | 26.83 | 6.97 | 0.260 | 74% |
| 173 | 279 | 22.51 | 5.39 | 0.239 | 76% |

[symbol]

indicates data missing or illegible when filed

Table 1: Catalyst activity of spent Amberlyst™ 15 compared to fresh catalyst evaluated in an FBR at room temperature, 1 wt. % formic acid and 1 wt. % hydrogen peroxide feed concentrations.

### Example 2

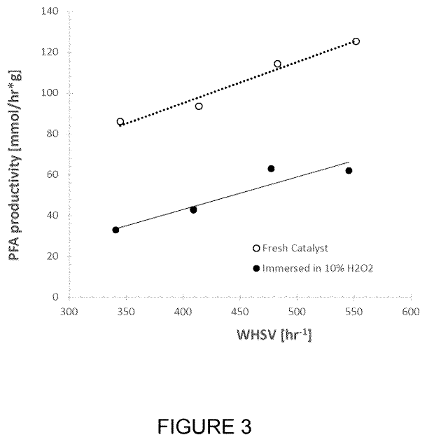

The stability of commercially available Amberlyst™ 15 (Sigma Aldrich 216380) as a catalyst for PFA synthesis was tested in a bench scale experiment by first measuring the PFA productivity (mmol/hr*g_{cat}) of fresh Amberlyst™ 15 for PFA synthesis in a fixed bed flow reactor at weight hourly space velocities ranging from about 250 to 550 hr¹⁻ for H₂O₂ and formic acid feeds streams, both 3 wt % (aq) before combining the two reactant feed streams at the entrance to the FBR. The tests were repeated using Amberlyst™ 15 which had been first exposed in a beaker to 10 wt % (aq) H₂O₂ solution for about 72 hours before testing its activity by repeating the aforementioned tests at various space velocities and at room temperature. The results in FIG. 3 show that Amberlyst™ 15 had lost roughly 62% of its activity (measured at WHSV=340 hr¹) after exposure to this relatively modest concentration of H₂O₂.

As can be seen from Examples 1 and 2 and FIG. 3, it is clear that Amberlyst™ 15 is not robust and is not stable for prolonged lengths of time required for commercial applications involving the use of concentrated hydrogen peroxide and or catalytic distillation reaction environment for the production of PFA.

Examples 3 to 8 are non-limiting examples of the present catalytic system. Examples 3, 4 and 6 are illustrative of Embodiment #1. The purpose of the Examples 3, 4 and 6 are to demonstrate the importance of the synergistic effect between the co-catalysts to improve the stability and activity of the catalyst for the production of PFA. Example 3 is an example of Embodiment #1, whereby the carrier is Al₂O₃, and co-catalyst #1 is Nb₂O₅, however, co-catalyst #2 is absent. In contrast, Examples 4 and 5 contains co-catalyst #2, (PdO) in addition to co-catalyst #1 (Nb₂O₅) which gives a superior result. Similarly, in Example 6, the catalyst contains the co-catalyst PdO but co-catalyst #1 is absent, giving poor performance compared to the catalyst of Examples 4 and 5.

### Example 3

A Nb₂O₅/Al₂O₃ solid acid catalyst was created from the impregnation of a Al₂O₃ catalyst carrier having a specific surface area of approximately 160 m²/g, with NbCl₅ in methanol solvent. The catalyst was recovered and rinsed and dried overnight in air at 150° C. The catalyst was calcined for 2 hours in air at 100° C. The Nb₂O₅ mass fraction was approximately 19 wt. %. The activity of the fresh catalyst was measured at room temperature in a fixed bed reactor with 5 wt. % formic acid and 5 wt. % hydrogen peroxide fed into the reactor at a space velocity (WHSV) of 660 hr¹. The average PFA productivity of 11 experiments with the fresh catalyst was 215.7 (mmol/hr*g_{cat}).

The fresh catalyst was exposed to 10 wt. % H₂O₂ (aqueous) solution for 48 hours. The catalyst was then recovered and its activity tested in the FBR at the same conditions for which the fresh catalyst was tested. The PFA productivity of the spent catalyst was found to be 129.1 (mmol/hr*g_{cat}) which represents roughly a 40% loss of activity. Significantly, an obvious discolouration of the spent catalyst was observed; it exhibited a distinct yellow colour which is associated with the formation of metal peroxo groups and the deactivation of the transition metal oxide catalyst due to loss of surface acidity. This result is very similar to the result of the Amberlyst™ 15, considering the exposure times were slightly different.

### Example 4

Example #4 follows Embodiment #1, wherein the catalyst carrier is Al₂O₃, co-catalyst #1 is Nb₂O₅ and co-catalyst #2 is PdO.

A PdO/Nb₂O₅/Al₂O₃ catalyst was created by first creating the approximately 19 wt. % Nb₂O₅/SiO₂ described in Example 3 following the procedure outlined in Example 3. The catalyst was then exposed to concentrated hydrogen peroxide 10 wt. % for 1 hours at room temperature, rinsed with deionized water and then dried at 100° C. overnight. The catalyst was then impregnated with Pd(II) acetate in acetone solution by dry impregnation, where the impregnation solution was added dropwise until it was completely absorbed by the catalyst and the acetone was allowed to evapourate for % hour. The concentration of the palladium precursor was selected such that the final mass fraction of palladium in the catalyst would be approximately 0.5 wt. %. The catalyst was dried in air overnight at 150° C., and then calcined in air at 300° C. for 4 hours. Fresh catalyst was loaded to a fixed bed reactor (FBR) and the catalyst activity was tested at room temperature, with 3 wt. % H₂O₂ and 3 wt. % formic acid aqueous feed streams feeding the reactor at the same flow rate, giving a space velocity (WHSV) of 878 hr⁻¹. The experiment was repeated 14 times giving an average PFA productivity of 130 (mmol/hr*g_{cat}). The catalyst was recovered and exposed to concentrated H₂O₂ (10 wt. % aq.) for 48 hours and the catalyst was re-installed into the reactor and the activity tests were repeated an additional 11 times. Due to the slight difference in catalyst mass, the space velocity was 947 hr⁻¹. The average PFA productivity for the 11 experiments was found to be 123 (mmol/hr*g_{cat}). A t-test was conducted, and it was concluded that this result was not statistically different at the 95% confidence level from the result obtained for the fresh catalyst, indicating no measurable catalyst deactivation had occurred.

### Example 5

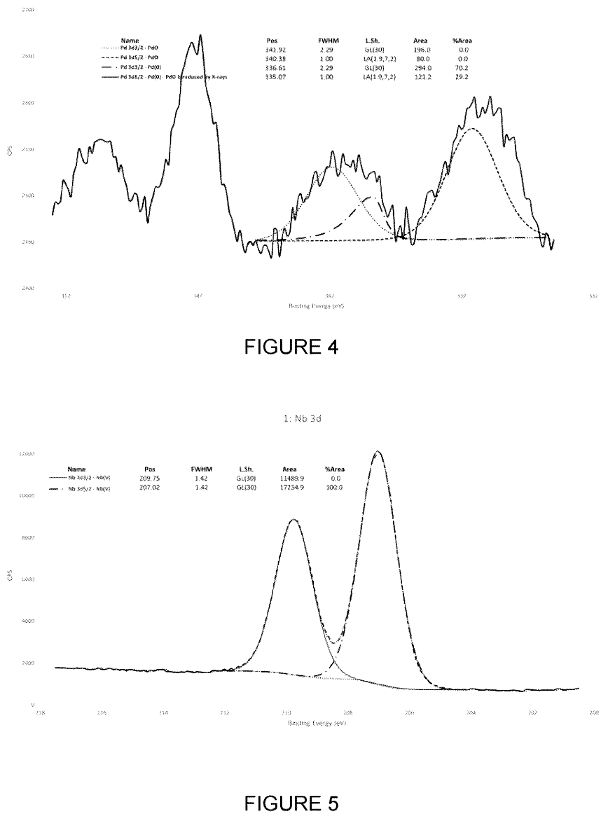

The nominal 0.5 wt. % PdO/Nb₂O₅/Al₂O₃ catalyst created in Example 4 was analyzed by low resolution and high resolution X-Ray photoelectron spectroscopy (XPS). XPS is a surface sensitive technique which reports the elemental composition near the surface of the catalyst to a depth of 7 to 10 nm. Inference on structure and bonding may be made based on observed shifts in the characteristic binding energies. The low resolution survey scans were carried out with an analysis area of 300 μm×700 μm and a pass energy of 160 eV. The elemental analysis obtained from the low resolution survey scan is outlined below. High resolution XPS analysis was carried out with an analysis area of 300 μm×700 μm and a pass energy of 20 eV; the spectra were charge corrected to the main line of C 1s spectrum set to 284.8 eV for adventitious carbon. The high resolution XPS of the Pd 3d spectra (FIG. 4) show that the palladium was primarily in the form PdO with some metallic palladium present. It is known that the X-ray bombardment may reduce PdO to metallic palladium. The high resolution XPS spectra of Nb 3d (see FIG. 5) confirm the presence of Nb₂O₅.

| element | at % |
|---|---|
| Al | 8.9 |
| C | 33 |
| Ca | 0.3 |
| Cl | 0 |
| F | 0 |
| N | 0 |
| Na | 0 |
| Nb | 11.2 |
| O | 46.3 |
| Pd | 0.2 |
| S | 0.3 |

### Example 6

A 0.5 wt. % PdO/Al₂O₃ catalyst was created by dry impregnation using a solution of Pd(II) acetate in acetone, which was added dropwise and the acetone allowed to evapourate for % hour. The catalyst was dried in air at 150° C. overnight and calcined in air at 300° C. for 4 hours. The catalyst activity was tested in a fixed bed flow reactor at room temperature with 3 wt. % H₂O₂ and 3 wt. % formic acid feed streams resulting in a space velocity in experiment ranging from 692 to 706 hr⁻¹. Although this space velocity was lower than that for the experiment with PdO/Nb₂O₅/Al₂O₃, the activity of the PdO/Al₂O₃ catalyst was lower than expected. The average PFA productivity of the catalyst was 86.7 (mmol/hr*g_{cat}) over 9 experiments. Significantly, the catalyst activity continued to decrease with each experiment, with the PFA productivity dropping as low as 66.3 (mmol/hr*g_{cat}). This result, in contrast to that of Example 3, illustrates that the presence of the Nb₂O₅ co-catalyst #1 promoted both the activity and the stability of the PdO co-catalyst.

While PdO and Nb₂O₅ gave good results as co-catalysts for this first Embodiment #1, the inventors contemplate that PdO could be replaced with oxides of {Ag, Pt, W, Ni, Rh} or by metal carbides of {Mo, V, W, Ti, Co} or metal nitrides of {W, Mo, Ti, V, Nb}. Similarly, the inventors contemplate that Nb₂O₅ could be replaced with transition metal oxides known to have high surface concentrations of hydroxyl groups such as {SiO₂, V₂O₅, TiO_{2}.}

Some examples of co-catalyst #2 which do not work well in a synergistic manner with Nb₂O₅ as co-catalyst #1 include {CoO, ZnO, Fe₂O₃, CuO}.

Examples 7 and 8 are illustrative of the Embodiment #2, where the catalyst carrier participates substantively in the overall catalytic reaction and is designated as co-catalyst #1, and which works synergistically with a second catalyst phase co-catalyst #2. In both examples, the catalyst carrier is a mesoporous Al₂O₃ support with nominal specific surface area of about 160 m²/g. In Example 7 co-catalyst #2 is Ag₂O while in Example 8 co-catalyst #2 is WO₃.

### Example 7

A Ag₂O/Al₂O₃ catalyst was created by dry impregnation by adding aqueous solutions of silver acetate dropwise to the Al₂O₃ carrier and allowing the solvent to evapourate for ½ hour. The catalyst was dried in air at 150° C. overnight and subsequently calcined in air at 300° C. for 4 hours. The nominal Ag mass fraction was approximately 2.5 wt. %. The catalyst was tested in a fixed bed reactor at room temperature in 3 separate experiments with 3 wt. % H₂O₂ and 3 wt. % formic acid solutions feeding the reactor resulting in a weight hourly space velocity (WHSV) of 1125 hr¹. The average PFA productivity was 135.3 (mmol/hr*g_{cat}). The catalyst was exposed to 10 wt. % H₂O₂ at room temperature overnight. The recovered catalyst was tested an additional 3 times at the same conditions of room temperature, with 3 wt. % H₂O₂ and 3 wt. % formic acid solutions feeding the reactor resulting in a space velocity (WHSV) of 1125 hr⁻¹. The average PFA productivity was found to be 133.5 (mmol/hr*g_{cat}).

### Example 8

A WO₃/Al₂O₃ catalyst was created by dry impregnation by adding aqueous solutions of sodium tungstate dihydrate dropwise to the Al₂O₃ carrier and allowing the solvent to evapourate for ½ hour. The catalyst was dried in air at 150° C. overnight and subsequently calcined in air at 300° C. for 4 hours. The nominal W mass fraction was 5 wt. %. The catalyst was tested in a fixed bed reactor at room temperature in 4 separate experiments with 3 wt. % H₂O₂ and 3 wt. % formic acid solutions feeding the reactor resulting in a space velocity (WHSV) of 878 hr⁻¹. The average PFA productivity was 118.1 (mmol/hr*g_{cat}). The catalyst was exposed to 10 wt. % H₂O₂ at room temperature overnight. The recovered catalyst was tested an additional 5 times at the same conditions of room temperature, with 3 wt. % H₂O₂ and 3 wt. % formic acid solutions feeding the reactor resulting in a space velocity (WHSV) of 878 hr⁻¹. The average PFA productivity was found to be 115.5 (mmol/hr*g_{cat}). The combination of these two materials results in synergistic benefits including improved activity and stability.

Based on Examples 3 to 8, the system of Embodiment #1 with PdO/Nb₂O₅/Al₂O₃, illustrates the best results. While the above have been carried out using Al₂O₃ as the catalyst carrier, it will be appreciated that other materials for example SiO₂, can be used as the catalyst carrier. A key feature of Embodiment #2, is that the catalyst carrier may exhibit catalytic sites such as Lewis or Bronsted acidity or basicity or otherwise interacts with co-catalyst #1 in a manner which has a substantive impact on the catalytic turnover. The inventors found that when mesoporous Al₂O₃ was used as the carrier, that oxides of Ag and W as co-catalyst #2 worked particularly well. Some examples of co-catalyst #2 materials which do not work well in Embodiment #2 in a synergistic manner with Al₂O₃ as co-catalyst #1 include {PdO, Nb₂O₅, MoO_{3}.}

In summary, in an embodiment the present disclosure provides a catalyst for the production of a percarboxylic acid. The catalyst comprises a mesoporous catalyst material having an external surface and internal pores and having inherent surface acidity or surface basicity, said mesoporous catalyst material being in a form of i) a particle or extrudate ranging in size from about 0.2 to about 25 mm, or ii) a monolith, or iii) one or more thin films having a thickness in a range from about 1 to 1000 μm. The mesoporous catalyst material has a specific surface area ranging from about 1 to about 10,000 m²/g. The catalyst includes a co-catalyst comprised of any one or combination of WO₃ and Ag₂O dispersed as particles or thin films onto the internal surface area and/or external surface area of the mesoporous catalyst material.

In an embodiment the mesoporous catalyst material has a specific surface area in a range from about 100 to about 600 m²/g, or a specific surface area in a range from about 150 to about 600 m²/g.

In an embodiment the mesoporous catalyst material possess either inherent surface acidity or inherent surface basicity is characterized in that it
i) provides active sites which can participate directly in the perhydrolysis reaction due to the inherent surface acidity or basicity of the mesoporous catalyst material or other unique imperfections, surface defects, functional groups or features, or
ii) creates adlineation sites at phase boundaries between the mesoporous catalyst material and the dispersed co-catalyst, or
iii) interacts with the co-catalyst to generate weak or strong metal support interactions at the reaction conditions, or
iv) any combination of i), ii) or iii).

In an embodiment the mesoporous catalyst material possess an inherent surface acidity characterized by a Hammet acidity of less than 3.

In an embodiment the mesoporous catalyst material is any one of Al₂O₃, WO₃, MgO, Nb₂O₅, TiO₂, MoO₃, Fe₂O₃, FeO, V₂O₅, MgO, ZnO, Y₂O₃, Sc₂O₃, ZrO₂, WO₃, W₂O₅, CeO₂, Ce₂O₃, Ta₂O₃, ZrWₓO_{y} (wherein x is 2 and y is 0.5 to 8), SnO₂, activate carbon, graphite and mixtures thereof.

In an embodiment the mesoporous material is Al₂O₃.

In an embodiment the mesoporous material is an aluminosilicate with the general formula Mⁿ⁺_{x/n}[(Al₂O₃)*(SiO₂)ₓ]*yH₂O, wherein M is a metal cation and n is the charge on the metal cation, typically ranging from 1 to 3, and x represents the silica to alumina ratio varying over a broad range from 1 to 500.

In an embodiment the mesoporous catalyst material is an acidic ion exchange resin or a basic ion exchange resin

In an embodiment the mesoporous catalyst material is an acidic or basic polymer bead.

In an embodiment the mesoporous catalyst material is a hydrotalcite with the general formula [M²⁺₁₋ₓM³⁺ₓ(OH)₂]^{x+}Aⁿ⁻_{x/n}*mH₂O wherein M²⁺ and M³⁺ are divalent and trivalent transition metal cations respectively and Aⁿ⁻ is an exchangeable anion, wherein x ranges from 0.2 to 0.4, and n is 1 or 2. The mesoporous catalyst material may be a mixture of hydrotalcite materials.

In an embodiment the mesoporous catalyst material is a semiconductor selected from the group consisting of Ta₂O₅, NaTaO₃, SrTiO₃, CdS, InVO₄, Mn₂O₃, Bi₂WO₆, BiVO₄, Ga₂O₃ and ZnGa₂O₄.

In an embodiment the any one or combination of co-catalyst WO₃ and Ag₂O may be dispersed as sub-micron sized particles.

The any one or combination of co-catalyst WO₃ and Ag₂O are dispersed as films having a thickness in a range from about 10 to about 300 μm.

In an embodiment the mesoporous catalyst material is mesoporous catalyst material may be in the form a particle or extrudate having a size in a range from about 0.2 to about 25 mm.

The mesoporous catalyst material may be in the form a particle or extrudate having a size in a range from about 3 mm to about 11 mm.

In an embodiment the mesoporous catalyst material is a mesoporous catalyst material may be in the form a film having a thickness in a range from about 1 to about 1000 μm.

The mesoporous catalyst material may be in the form a film having a thickness in a range from about 5 to about 300 μm.

The mesoporous catalyst material may be in the form a film having a thickness in a range from about 10 to about 200 μm.

The percarboxylic acid may be performic acid.

In an embodiment the present disclosure also provides a catalyst for the production of percarboxylic acids, comprising:
a mesoporous catalyst carrier material having an external surface and interior pores extending therethrough;
at least first and second distinct co-catalyst materials which are dispersed onto the external surface and/or into the interior pores of the mesoporous catalyst material either as particles or in one or more thin films;
the first co-catalyst being derivatized to produce metal peroxo groups;
the at least second co-catalyst selected to interact with said first co-catalyst by providing active sites for the chemisorption of the parent carboxylic acid and or by providing surface features to facilitate the surface mobility and migration of surface active oxygen species.

The metal peroxo groups are capable of storing and releasing surface active oxygen to the catalyst surface. The product, peracid is created from the concerted addition of surface active oxygen species to the parent acid, which is chemisorbed on an active site of co-catalyst 2, which then desorbs from the active site of co-catalyst 2, thereby releasing the product from the catalyst while regenerating the active sites for the next catalytic cycle. Similarly, the presence of concentrated hydrogen peroxide regenerates the metal peroxo group, for the next catalytic cycle.

The first co-catalyst derivatized to produce metal-peroxo groups may be any one or combination of Nb₂O₅, SiO₂, TiO₂, MoO₂, Al₂O₃, Fe₂O₃, V₂O₅, MgO, ZnO, Y₂O₃, Sc₂O₃, ZrO₂, W₂O₅, WO₃, CeO₂ and Ta₂O₃.

In an embodiment the co-catalyst derivatized to produce metal peroxo groups is Nb₂O₅.

The at least a second co-catalyst is any one or combination of transition metals or oxides of transition metals from the group consisting of Ag, Pt, Pd, W, Ni, Rh, Ru, Au, Ir, Os, Re, Ta, Hf, Zr, Ti, V, Cr, Mo, Mn, Co, Zn, Fe and Cu.

The at least a second co-catalyst is any one or combination of metal carbides of the transition metals selected from the group {M, V, W, Ti, Co} or metal nitride of the transition metals selected from the group consisting of W, Mo, Ti, V and Nb.

In an embodiment the at least a second co-catalyst is PdO.

In an embodiment the mesoporous catalyst carrier is
selected from the group consisting of SiO₂, Al₂O₃, TiO₂, MgO, WO₃, MnO₂, MoO₂, ZnO, Fe₂O₃, V₂O₅, Y₂O₃, Sc₂O₃, ZrO₂, CeO₂, Ce₂O₃ and Ta₂O₃, or
is an aluminosilicate with the general formula Mⁿ⁺_{x/n}[(Al₂O₃)*(SiO₂)ₓ]*yH₂O, wherein M is a metal cation and n is the charge on the metal cation ranging from 1 to 3, and x represents the silica to alumina ratio which can vary over a broad range from 1 to 500; or
is a hydrotalcite material with the general formula [M²⁺₁₋ₓM³⁺ₓ(OH)₂]^{x+}Aⁿ⁻_{x/n}*mH₂O wherein M²⁺ and M³⁺ are divalent and trivalent transition metal cations respectively and Aⁿ⁻ is an exchangeable anion, wherein x ranges from 0.2 to 0.4, n is typically 1 or 2; or any combination thereof.

In an embodiment the mesoporous catalyst carrier is either SiO₂ or Al₂O₃.

### REFERENCES

1. American Water Works Association, Water Utility Disinfection Survey Report, Cornwell Engineering Group, April 2018
2. R. J. Bull et al., Toxicology, 2011, 286, (1-3), 1.
3. T. Karpova et al., Water. Sci. Technol., 2013, 68(9), 2090.
4. R. Maffettone et al., WEFTEC 2018 Proceedings
5. R. Aksela and I. Renvall, “Process for the Preparation of Peroxy Acids”, PCT/F12006/000303
6. V. Rocher and S. Azimi, “Effectiveness of Disinfecting Wastewater Treatment Plant Discharges: Case of Chemical Disinfection using Performic Acid”, IWA Publishing (2021), pg. 61.
7. W. K. O'Keefe, Ind. Eng. Chem. Res., 2007, 46(3), 716
8. R. Taylor and R. Krishna, Chem. Eng. Sci., 2000, 55, 5183.
9. S. Pohjanvesi et al., US 2002/0177732 A1 (2002), “Process for the Production of Percarboxylic Acid
10. R. Balasubramanian et al., “Performic Acid On-site Generator and Formulator”, AU2019208211 B2
11. T. Aksela and T. Mattila, “A method of preparing aqueous solutions containing performic acid as well as their use”, EP0751933B1
12. S. Leveneur et al., Ind. Eng. Chem. Res., 2012, 51, 13999.
13. F. Ebrahimi et al. Chem. Eng. J., 2011, 167, 713.
14. F. Ebrahimi et al., Chem. Eng. J, 2012, 179, 312
15. S. Talwakar and S. Mahajani, Appl. Catal. A, 2006, 302, 140.
16. W. Nicol and E. DuToit, Chem. Eng. Process., 2004, 43, 1539.
17. A. Chakrabarti and M. M. Sharma, “Cation Exchange Resins as Catalysts”, React. Funct. Polym, 1993, 20, 1.
18. K. Tanabe, “Solid Acids and Bases: Their Catalytic Properties”, Elsevier (2012).
19. K. Tanabe, Mat. Chem. Phys. 1987, 17, 217.

1. A catalyst for the production of a percarboxylic acid, comprising:
a mesoporous catalyst material having an external surface and internal pores and having inherent surface acidity or surface basicity, said mesoporous catalyst material being in a form of
i) a particle or extrudate ranging in size from about 0.2 to about 25 mm, or
ii) a monolith, or
iii) one or more thin films having a thickness in a range from about 1 to 1000 μm;
the mesoporous catalyst material having a specific surface area ranging from about 1 to about 10,000 m²/g; and
a co-catalyst comprised of any one or combination of WO₃ and Ag₂O dispersed as particles or thin films onto the internal surface area and/or external surface area of the mesoporous catalyst material.

2. The catalyst according to claim 1, wherein said mesoporous catalyst material has a specific surface area in a range from about 100 to about 600 m²/g.

3. The catalyst according to claim 1, wherein said mesoporous catalyst material has a specific surface area in a range from about 150 to about 600 m²/g.

4. The catalyst according to claim 1, wherein said mesoporous catalyst material possessing either inherent surface acidity or inherent surface basicity is characterized in that it
i) provides active sites which can participate directly in the perhydrolysis reaction due to the inherent surface acidity or basicity of the mesoporous catalyst material or other unique imperfections, surface defects, functional groups or features, or
ii) creates adlineation sites at phase boundaries between the mesoporous catalyst material and the dispersed co-catalyst, or
iii) interacts with the co-catalyst to generate weak or strong metal support interactions at the reaction conditions, or
iv) any combination of i), ii) or iii).

5. The catalyst according to claim 1, wherein said mesoporous catalyst material possesses an inherent surface acidity characterized by a Hammet acidity of less than 3.

6. The catalyst according to claim 1, wherein said mesoporous catalyst material is any one of Al₂O₃, WO₃, MgO, Nb₂O₅, TiO₂, MoO₃, Fe₂O₃, FeO, V₂O₅, MgO, ZnO, Y₂O₃, Sc₂O₃, ZrO₂, WO₃, W₂O₅, CeO₂, Ce₂O₃, Ta₂O₃, ZrWₓO_{y} (wherein x is 2 and y is 0.5 to 8), SnO₂, activate carbon, graphite and mixtures thereof.

7. The catalyst according to claim 1, wherein said mesoporous material is Al₂O₃.

8. The catalyst according to claim 1, wherein said mesoporous catalyst material is an aluminosilicate with the general formula Mⁿ⁺_{x/n}[(Al₂O₃)*(SiO₂)ₓ]*yH₂O, wherein M is a metal cation and n is the charge on the metal cation, typically ranging from 1 to 3, and x represents the silica to alumina ratio varying over a broad range from 1 to 500.

9. The catalyst according to claim 1, wherein said mesoporous catalyst material is an acidic ion exchange resin or a basic ion exchange resin

10. The catalyst according to claim 1, wherein said mesoporous catalyst material is an acidic or basic polymer bead.

11. The catalyst according to claim 1, wherein said mesoporous catalyst material is a hydrotalcite with the general formula [M²⁺₁₋ₓM³⁺ₓ(OH)₂]^{x+}Aⁿ⁻_{x/n}*mH₂O wherein M²⁺ and M³⁺ are divalent and trivalent transition metal cations respectively and Aⁿ⁻ is an exchangeable anion, wherein x ranges from 0.2 to 0.4, and n is 1 or 2.

12. The catalyst according to claim 11, wherein mesoporous catalyst material is a mixture of hydrotalcite materials.

13. The catalyst according to claim 1 wherein said mesoporous catalyst material is a semiconductor selected from the group consisting of Ta₂O₅, NaTaO₃, SrTiO₃, CdS, InVO₄, Mn₂O₃, Bi₂WO₆, BiVO₄, Ga₂O₃ and ZnGa₂O₄.

14. The catalyst according to claim 1, wherein said of any one or combination of co-catalyst WO₃ and Ag₂O are dispersed as sub-micron sized particles.

15. The catalyst according to claim 1, wherein said of any one or combination of co-catalyst WO₃ and Ag₂O are dispersed as films having a thickness in a range from about 10 to about 300 μm.

16. The catalyst according to claim 1, wherein said mesoporous catalyst material in the form a particle or extrudate having a size in a range from about 0.2 to about 25 mm.

17. The catalyst according to claim 1, wherein said mesoporous catalyst material in the form a particle or extrudate having a size in a range from about 3 mm to about 11 mm.

18. The catalyst according to claim 1, wherein said mesoporous catalyst material in the form a film having a thickness in a range from about 1 to about 1000 μm.

19. The catalyst according to claim 1, wherein said mesoporous catalyst material in the form a film having a thickness in a range from about 5 to about 300 μm.

20. The catalyst according to claim 1, wherein said mesoporous catalyst material in the form a film having a thickness in a range from about 10 to about 200 μm.

21. The catalyst according to claim 1, wherein said percarboxylic acid is performic acid.

22. A catalyst for the production of percarboxylic acids, comprising:
a mesoporous catalyst carrier material having an external surface and interior pores extending therethrough;
at least first and second distinct co-catalyst materials which are dispersed onto the external surface and/or into the interior pores of the mesoporous catalyst material either as particles or in one or more thin films;
said first co-catalyst being derivatized to produce metal peroxo groups;
said at least second co-catalyst selected to interact with said first co-catalyst by providing active sites for the chemisorption of the parent carboxylic acid and/or by providing surface features to facilitate the surface mobility and migration of surface active oxygen species.

23. The catalyst according to claim 22, wherein the first co-catalyst derivatized to produce metal-peroxo groups is any one or combination of Nb₂O₅, SiO₂, TiO₂, MoO₂, Al₂O₃, Fe₂O₃, V₂O₅, MgO, ZnO, Y₂O₃, Sc₂O₃, ZrO₂, W₂O₅, WO₃, CeO₂ and Ta₂O₃.

24. The catalyst according to claim 22, wherein the co-catalyst derivatized to produce metal peroxo groups is Nb₂O₅.

25. The catalyst according to claim 22, wherein said at least a second co-catalyst is any one or combination of transition metals or oxides of transition metals from the group consisting of Ag, Pt, Pd, W, Ni, Rh, Ru, Au, Ir, Os, Re, Ta, Hf, Zr, Ti, V, Cr, Mo, Mn, Co, Zn, Fe and Cu.

26. The catalyst according to claim 22, wherein said at least a second co-catalyst is any one or combination of metal carbides of the transition metals selected from the group {M, V, W, Ti, Co} or metal nitride of the transition metals selected from the group consisting of W, Mo, Ti, V and Nb.

27. The catalyst according to claim 22, wherein said at least a second co-catalyst is PdO.

28. The catalyst according to claim 22, wherein said mesoporous catalyst carrier is
selected from the group consisting of SiO₂, Al₂O₃, TiO₂, MgO, WO₃, MnO₂, MoO₂, ZnO, Fe₂O₃, V₂O₅, Y₂O₃, Sc₂O₃, ZrO₂, CeO₂, Ce₂O₃ and Ta₂O₃, or
is an aluminosilicate with the general formula Mⁿ⁺_{x/n}[(Al₂O₃)*(SiO₂)ₓ]*yH₂O, wherein M is a metal cation and n is the charge on the metal cation ranging from 1 to 3, and x represents the silica to alumina ratio which can vary over a broad range from 1 to 500; or
is a hydrotalcite material with the general formula [M²⁺₁₋ₓM³⁺ₓ(OH)₂]^{x}+Aⁿ⁻_{x/n}*mH₂O wherein M²⁺ and M³⁺ are divalent and trivalent transition metal cations respectively and Aⁿ⁻ is an exchangeable anion, wherein x ranges from 0.2 to 0.4, n is typically 1 or 2; or any combination thereof.

29. The catalyst according to claim 21, wherein said mesoporous catalyst carrier is SiO₂.

30. The catalyst according to claim 21, wherein said mesoporous catalyst carrier is Al₂O₃.
